(19) United States
(12) Patent Application Publication
(10) Pub. No.: US 2025/0082805 A1
(43) Pub. Date: Mar. 13, 2025

(54) FILTER SUPPORT SYSTEM HAVING AN ANTI-MICROBIAL COMPONENT

(71) Applicant: Roger Richter, Elkhorn, NE (US)

(72) Inventors: Roger Richter, Elkhorn, NE (US); Shawn VanFossen, Auburn, KS (US)

(21) Appl. No.: 18/806,520

(22) Filed: Aug. 15, 2024

Related U.S. Application Data
(63) Continuation-in-part of application No. 17/010,753, filed on Sep. 2, 2020.

(51) Int. Cl. A61L 2/238; B01D 46/00

(52) U.S. Cl. CPC A61L 2/238; B01D 46/0005

(57) ABSTRACT

A filter support system may include, but is not limited to: an anti-microbial mesh; and a frame configured to at least partially retain a filter such that at least a portion of fluid flow through the filter interacts with the anti-microbial mesh. The anti-microbial mesh may be constructed at least partially from a copper-containing material.

### CROSS REFERENCE TO RELATED APPLICATION

The present application claims priority to U.S. application Ser. No. 17/010,753, filed Sep. 2, 2020, entitled FILTER SUPPORT SYSTEM HAVING AN ANTI-MICROBIAL COMPONENT, naming Roger Richter and Shawn VanFossen as inventors, which is incorporated herein by reference in the entirety to the extent not inconsistent herewith.

### FIELD OF THE INVENTION

The invention relates generally to the field of residential, commercial, industrial and/or automotive filtration systems.

### BRIEF DESCRIPTION OF THE DRAWINGS

The numerous advantages of the disclosure may be better understood by those skilled in the art by reference to the accompanying figures in which:

FIG. 1 shows a filter support system having an anti-microbial component;

FIG. 2 shows a filter support system having an anti-microbial component;

FIG. 3 shows a filter support system having an anti-microbial component;

FIG. 4 shows a filter support system having an anti-microbial component;

FIG. 5 shows a filter support system having an anti-microbial component;

FIG. 6 shows an anti-microbial function testing system;

FIG. 7 shows configurations of an anti-microbial mesh;

FIG. 8 shows a testing parameter matrix;

FIG. 9 shows a chart detailing pressure drop measurements;

FIG. 10 shows a chart detailing bacteriophage reduction;

FIG. 11 shows a chart detailing bacteriophage reduction;

FIG. 12 shows a chart detailing bacteriophage reduction; and

FIG. 13 shows a chart detailing bacteriophage reduction.

### DETAILED DESCRIPTION

Reference will now be made in detail to various embodiments of the invention, examples of which are illustrated in the accompanying drawings. While the invention will be described in conjunction with the following embodiments, it will be understood that the descriptions are not intended to limit the invention to these embodiments. On the contrary, the invention is intended to cover alternatives, modifications and equivalents that may be included within the spirit and scope of the invention. Furthermore, in the following detailed description, numerous specific details are set forth in order to provide a thorough understanding of the present invention. However, it will be readily apparent to one skilled in the art that the present invention may be practiced without these specific details. In other instances, well-known methods, procedures, components and materials have not been described in detail so as not to unnecessarily obscure aspects of the present invention.

The technical proposal(s) of embodiments of the present invention will be fully and clearly described in conjunction with the drawings in the following embodiments. It will be understood that the descriptions are not intended to limit the invention to these embodiments. Based on the described embodiments of the present invention, other embodiments can be obtained by one skilled in the art without creative contribution and are in the scope of legal protection given to the present invention.

Furthermore, all characteristics, measures or processes disclosed in this document, except characteristics and/or processes that are mutually exclusive, can be combined in any manner and in any combination possible. Any characteristic disclosed in the present specification, claims, Abstract and Figures can be replaced by other equivalent characteristics or characteristics with similar objectives, purposes and/or functions, unless specified otherwise.

The advantages of the present invention will become readily apparent from the detailed description of various embodiments below.

Bacteria, yeasts, viruses, and other potential bio-contaminants may be rapidly deactivated when brought into contact with or retained on surfaces including various metal components and/or coatings. As such, these materials may be employed as an anti-microbial and/or disinfecting component in various products. For example, copper and its alloys (brasses, bronzes, cupronickel, copper-nickel-zinc, and the like), silver, or any human-touch safe anti-microbial coating may be employed on high-touch surfaces, such as door handles, bathroom fixtures, or bed rails, in attempts to curb infectious transmissions.

In a further example, anti-microbial structures may be employed in concert with various heating, ventilation, and air conditioning (HVAC) to remove or deactivate such bio-contaminants present in an ambient environment (e.g. homes, businesses, schools, hospitals, arenas, concert halls, theaters, schools, gymnasiums, exposition/convention centers, shopping centers, airports, industrial facilities, and the like) in which large numbers of patrons or personnel may periodically visit and that, in order to create/maintain a clean environment for such individuals, should be sanitized to avoid transmission of communicable diseases

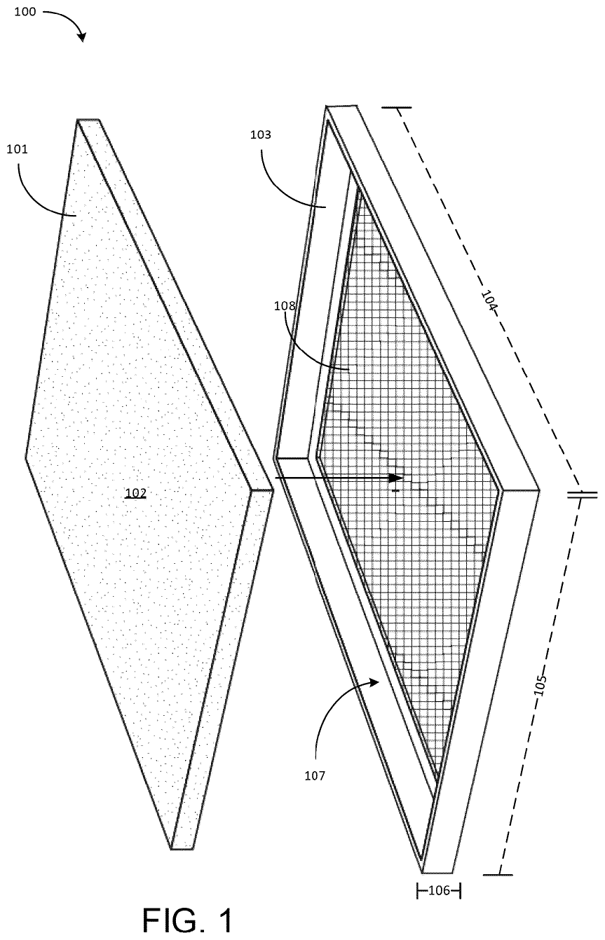

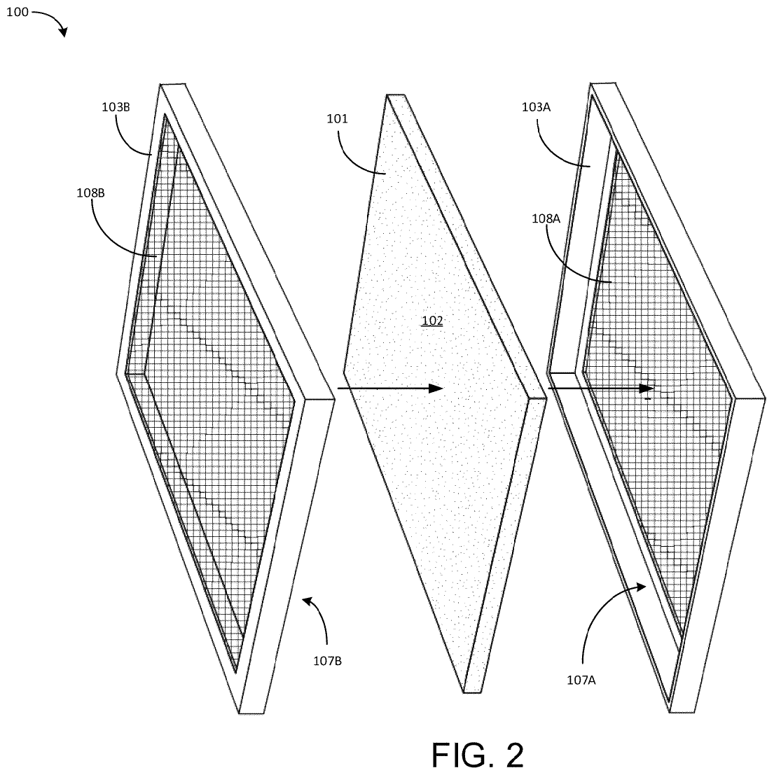

Referring to FIGS. 1-2, a system 100 for filtering and deactivation of bio-contaminants from an ambient environment is shown. The system 100 may include at least one filter 101. The filter 101 may include a membrane 102 constructed of a material (e.g. a cloth, fabric, plastic fiber (e.g. fiberglass), electrostatic, and the like) configured to permit fluid flow (e.g. airflow) through the membrane 102 while filtering and/or retaining particulate materials entrained within the fluid flow so as to purify the fluid flow. For example, the filter 101 may be selected from any number of original equipment manufacturer (OEM) HVAC air filters such as those manufactured by AIRx™, Honeywell™, Filtrete™, and the like.

The system 100 may further include at least one frame 103. The frame 103 may be sized such that it has a height 104, width 105, and depth 106 defining a recess 107 large enough to receive the filter 101 at least partially within the frame 103. Such a configuration may allow for the insertion of both the frame 103 and the filter 101 into a standard HVAC or other fluid flow conduit port (e.g. a residential or commercial HVAC filter slot).

The system 100 may further include an anti-microbial mesh 108. The anti-microbial mesh 108 (e.g. a network of interlaced fibers and/or wire portions, perforated sheets, and the like) may be constructed at least partially of an anti-microbial material. In one embodiment, the anti-microbial mesh 108 may be at least partially constructed from an anti-microbial metal. In another embodiment, the anti-microbial mesh 108 may be at least partially constructed from a copper-containing material. In another embodiment, the anti-microbial mesh 108 may be at least partially constructed of copper metal. In another embodiment, the anti-microbial mesh 108 may be at least partially constructed of a copper alloy. In another embodiment, the anti-microbial mesh 108 may be at least partially constructed of substrate at least partially coated with an anti-microbial coating.

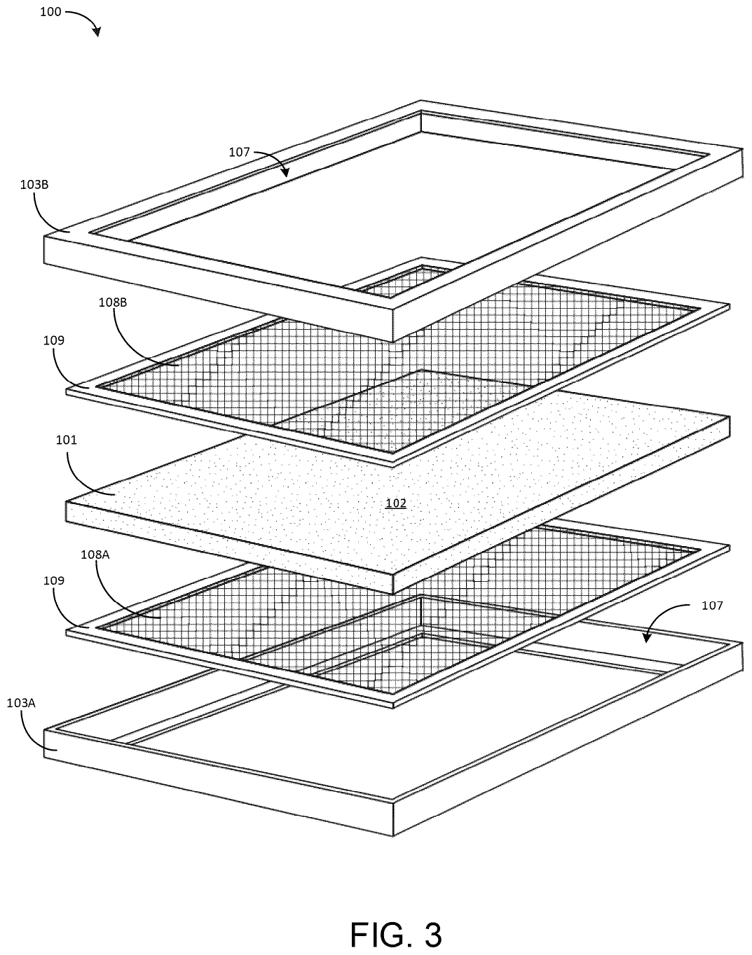

As shown in FIGS. 1 and 2, the anti-microbial mesh 108 may be directly coupled the frame 103 (e.g. via mechanical coupling, adhesive, welding, and the like). Alternately, as shown in FIG. 3, the anti-microbial mesh 108 may be coupled (e.g. via mechanical coupling, adhesive, welding, and the like) to a subframe 109 which, in turn, may be receivable within the recess 107 defined by a frame 103.

Referring again to FIG. 1, the system 100 may include a single frame 103 configured to receive the filter 101. The frame 103 may further include at least one retention mechanism (not shown) configured to retain the filter 101 at least partially within the frame 103. For example, the frame 103 may include one or more clips, straps, hooks, frictional contact portions, or other structures configured to retain the filter 101 at least partially within the frame 103.

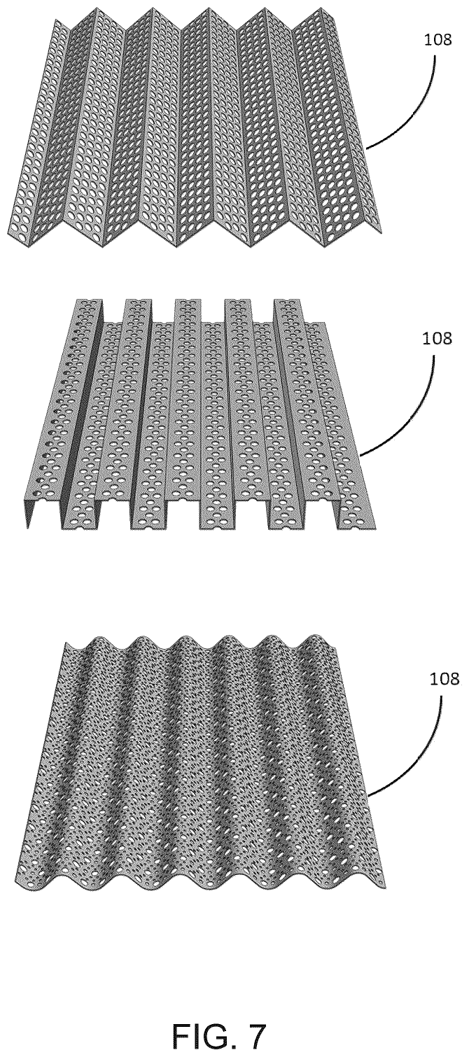

Referring to FIGS. 2 and 3, the system 100 may include at first frame portion 103A and a second frame portion 103B, including (e.g. receiving or coupled to) a first anti-microbial mesh portion 108A and a second anti-microbial mesh portion 108B, respectively, configured to cooperatively receive and/or retain the filter 101. Such a dual-frame, dual-mesh configuration may provide for increased surface area for interaction of fluid flow with the anti-microbial material of the first anti-microbial mesh portion 108A and the second anti-microbial mesh portion 108B. In another example, the anti-microbial mesh 108 may have a pleated, corrugated, or other three-dimensional configuration, to increase the effective surface area available for interaction with the fluid flow relative to the area defined by the frame 103. Various exemplary configurations of a pleated/corrugated anti-microbial mesh 108 (e.g., a repeating V-shaped, square-shaped, or wave-shaped configure, etc.) are shown in FIG. 7.

In one embodiment, the first frame portion 103A may define a recess 107A configured to at least partially receive the filter 101; the second 103B may define a recess 107B configured to at least partially receive both the first frame portion 103A and the filter 101 (e.g. inset at least partially within the second 103B). In another embodiment, the first frame portion 103A and the second 103B may be disposed adjacent to the filter 101 so as to sandwich the filter 101 between the first anti-microbial mesh portion 108A and the second anti-microbial mesh portion 108B. The first frame portion 103A and the second 103B may then be coupled together to retain the filter 101 therebetween.

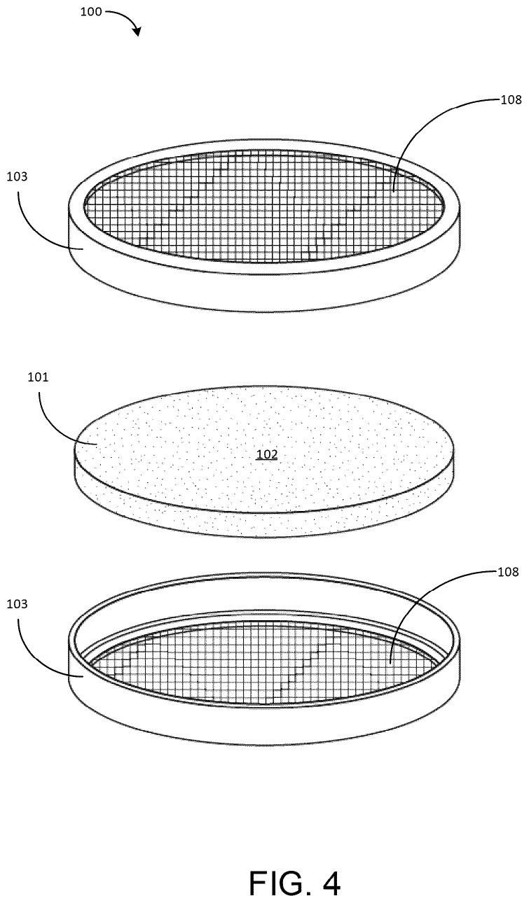

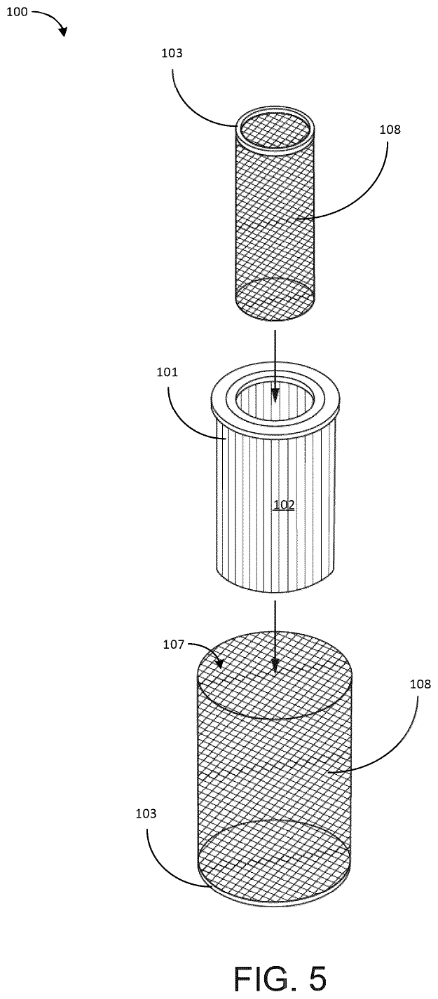

As shown in FIGS. 1-3, the frame 103 and/or the anti-microbial mesh 108 may have a rectilinear form factor substantially corresponding to a form factor of the filter 101. However, as shown in FIGS. 4 and 5, the frame 103 and/or the anti-microbial mesh 108 may be configured to correspond to a filter 101 having any form factor such that fluid flow through the filter 101 will interact with the anti-microbial mesh 108. For example, as shown in FIG. 4, the frame 103 and/or the anti-microbial mesh 108 may have a circular configuration so as to receive a circular filter 101. Alternately, as shown in FIG. 5, the frame 103 may support an anti-microbial mesh 108 having an at least partially cylindrical configuration so as to correspond to a cylindrical filter 101.

The anti-microbial mesh 108 may define a plurality of openings through which fluid flow may pass from one side of the anti-microbial mesh 108 to the other when the anti-microbial mesh 108 is placed in the path of the fluid flow. In various examples, the surface area sizing of the anti-microbial mesh 108 may be configured so as to regulate the degree of surface interaction between the fluid flow and the anti-microbial mesh 108 in order to provide a required residence time that the fluid flow is in contact with the anti-microbial mesh 108 so as to enable the treatment of the fluid flow via the anti-microbial properties of the anti-microbial mesh 108. This required residence time may be balanced with the overall flow rate of the fluid flow such that the anti-microbial treatment of the fluid flow is effective while maintaining sufficient throughput of fluid flow to effectively utilize the fluid flow medium (e.g., to heat or cool a space).

An open-area percentage of the anti-microbial mesh 108 may be defined as the percentage of the area of the anti-microbial mesh 108 taken up by the openings of the anti-microbial mesh 108 relative to the overall area defined by the anti-microbial mesh 108. For example, an anti-microbial mesh 108 corresponding to a standard 24-inch by 18-inch filter 101 may have a total area of 432 in² and an open-area percentage of 30%. In such an example, the surface area of the anti-microbial mesh 108 may be approximately 302 in² and the area of the openings in the anti-microbial mesh 108 may be approximately 130 in². In various examples, the anti-microbial mesh 108 may have an open-area percentage of from 10%-50%. More specifically, the anti-microbial mesh 108 may have an open-area percentage of from 20%-40%. Even more specifically, the anti-microbial mesh 108 may have an open-area percentage of approximately 30%.

In one example, the anti-microbial mesh 108 may be a 100-type mesh (e.g., a mesh screen having 100 openings per linear inch or 10,000 openings per square inch) having a 30% open-area percentage. Such a configuration may provide a balance of fluid flow/mesh contact surface area, residence time to fluid flow rate. It other examples, the sizing and spacing of the fiber or wire members of the mesh screen, and number of openings of anti-microbial mesh 108 may be adjusted to account for environments having higher bacterial loads and requiring more interaction of the fluid flow with the anti-microbial mesh 108 (resulting in either a slower fluid flow rate or requiring additional fluid pumping pressure) or lower bacterial loads requiring less interaction of the fluid flow with the anti-microbial mesh 108 (allowing for greater fluid flow rate and more efficient fluid pumping).

In various experimental examples, copper anti-microbial mesh 108 having a corrugated structure, a mesh size of 100, and a 30% open area (the “Test Mesh”) was employed. Airborne microbial reduction tests were conducted using: 1) a Filtrete® 2500 MPR filter (the “Test Filter”) as the filter 101; 2) the Test Filter and a layer of the Test Mesh on the downstream side of the Test Filter; and 3) the Test Filter, a first layer of the Test Mesh on the downstream side of the Test Filter and a second layer of the Test Mesh on the upstream side of the Test Filter.

Testing was conducted in a custom single pass bioaerosol challenge system constructed from PVC tubing at ARE Labs. The efficacy of the copper material was assessed via an upstream and downstream sampling method to evaluate viable challenge bioaerosol concentration (pfu/L). Comparison of the upstream and downstream samples yielded the single-pass efficiency in terms of the percent and LOG reduction of the bioaerosol challenge trials.

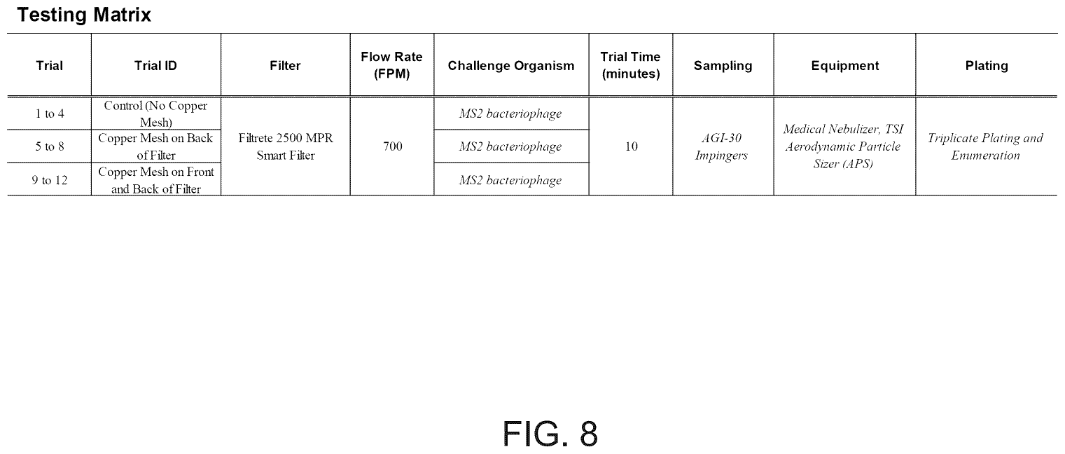

Testing was conducted to evaluate the single-pass reduction capabilities of the Test Mesh against a single viral sRNA bacteriophage which was MS2 bacteriophage. The material was tested at a flow rate of 700 ft/min. Referring to FIG. 8, the testing matrix is described:

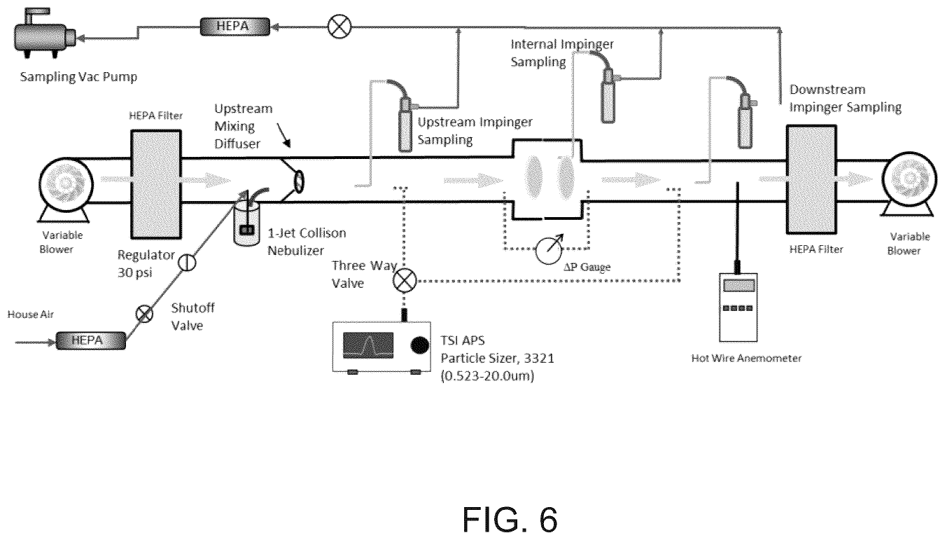

A bioaerosol testing system was constructed in order to conduct testing on the copper mesh material. The test system was assembled using 4″ PVC, impingers, in-line exhaust blowers, HEPA filters, and vacuum pumps. The system features sampling probes upstream and downstream of the device as well as three (3) separate locations for flow rate measurements. The system is primarily composed of 4″ PVC pipe, however a 3 ft long section of 2″ PVC is integrated into the inlet of the system. This was done to increase the airflow velocity within this section for more accurate flow rate measurements. FIG. 6 illustrates a flow diagram of an exemplary test system.

Test bioaerosols were disseminated using a medical nebulizer driven by HEPA filtered house air supply at 30 psi. A pressure regulator allowed for control of disseminated particle size, use rate, and sheer force generated within the nebulizer.

Two (2) AGI-30 impingers were used for bioaerosol sample collection for all trials conducted. The impingers were filled with 20 ml of Phosphate Buffered Saline (PBS) solution for collection of the bioaerosols. The impingers were then serially diluted and plated for direct enumeration of plaque or colony forming units (PFU/CFU).

The impinger flow vacuum source was maintained using a valved Emerson 1/3 hp rotary vane vacuum pump (Emerson Electric, St. Louis, MO) equipped with a 0-30 inHg vacuum gauge (WIKA Instruments, Lawrenceville, GA). The pump was operated at a negative pressure of 18 inches of Hg during all characterization and test sampling to assure critical flow conditions. The AGI-30 impingers sample at a flow rate of 12.5 LPM. Impingers sampled under critical flow conditions with vacuum>−18.0 inHg.

Prior to nebulization, the system flow was turned on and the variable blowers were adjusted to the desired flow rates as measured by a Testo 405i hotwire anemometer. Airflow measurements were taken at 3 different locations within the test system and compared to ensure uniform flow throughout the system. Flow measurements were taken at least twice before proceeding with testing in order to ensure no variations in flow rate. Mixing plates were integrated upstream and downstream of the material in order to ensure well mixed aerosols for a representative sample.

At the initiation of testing, a Hart mini Hi-Flo medical nebulizer was turned on and operated at 30 psi for the duration of each trial. The nebulizer was allowed to operate for at least 30 seconds prior to the initiation of sampling. Tests were conducted in sequence with the nebulizer turned off between test runs.

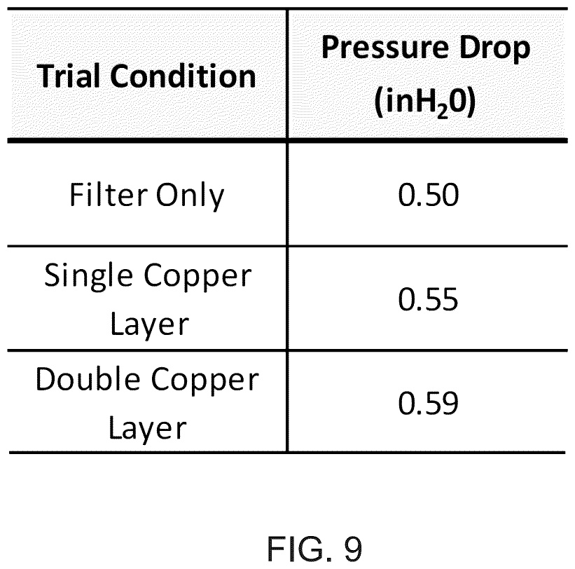

After the nebulizer operated for 30 seconds, both of the AGI-30 impingers were turned on at the same time at the up and down stream sampling locations. Impingers sampled for a period of ten (10) minutes with the nebulizer operating continuously. At the conclusion of each test the blowers remained on while impinger samples were collected and the impingers were cleaned for the next trial. As shown in FIG. 9, for all conducted trials pressure drop was recorded across the face of the filter and copper mesh.

Impinger and stock biological cultures were serially diluted and plated in triplicate (multiple serial dilutions) using a standard small drop assay technique onto tryptic soy agar plates. The plated cultures were incubated for 24 hours and enumerated and recorded.

At the conclusion of all testing, the nebulizer was cleaned and filled with 35% Hydrogen Peroxide. The peroxide was nebulized for approximately fifteen minutes while the variable speed blowers operated continuously. The nebulizer was then turned off while the blowers continued to run through the system for an additional 30 minutes in order to ensure all hydrogen peroxide was removed from the system.

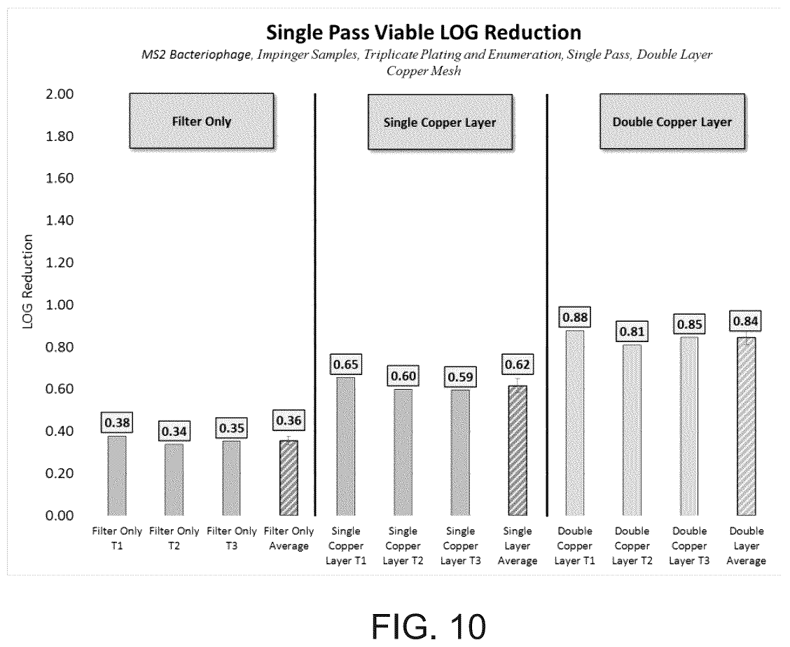

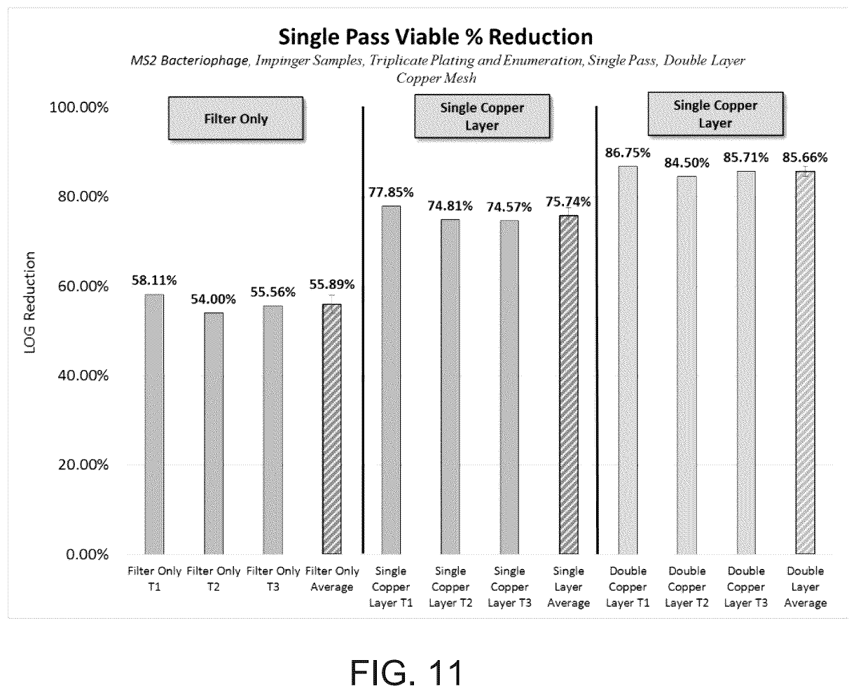

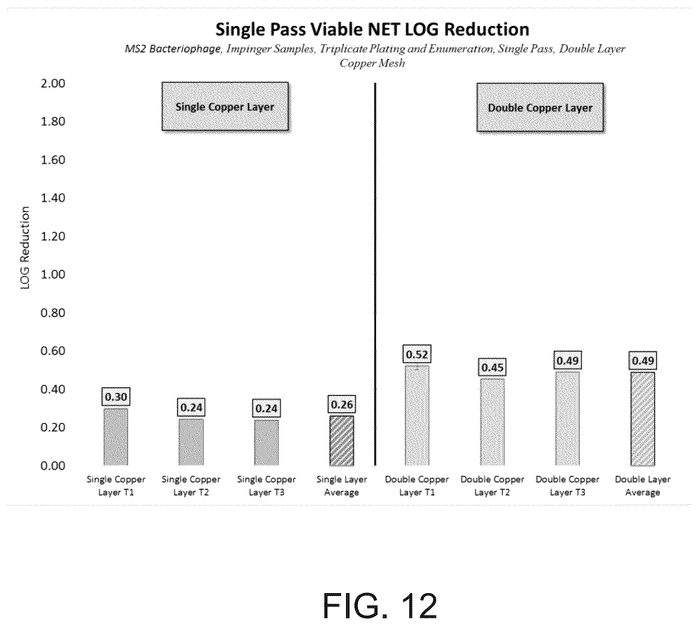

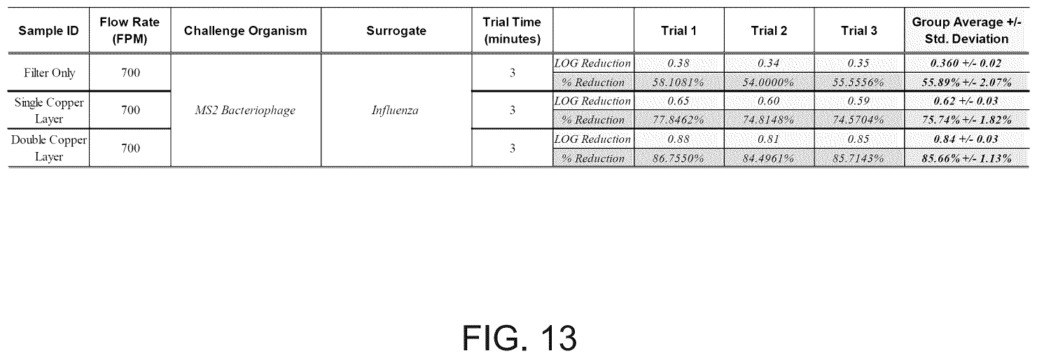

Referring to FIGS. 10-13, when tested with the Test Filter only in place there was an average 0.36+/−0.02 LOG reduction of MS2 bacteriophage. This testing was conducted with no Test Mesh in place. When tested with a single layer of Test Mesh on the downstream side of the Test Filter, an average 0.62+/−0.03 LOG reduction MS2 bacteriophage was achieved. When an additional layer of Test Mesh was added to the upstream side of the filter, testing showed a 0.84+/−0.03 LOG reduction of MS2 bacteriophage. These results indicate that the Test Mesh does have an effect on microbial reduction. When two (2) layers of copper mesh were used the reduction efficacy increased to 0.84 LOG compared to 0.36 LOG with only the filter in use.

When the Test Filter-only reductions were subtracted, the copper mesh showed an average net LOG reduction of 0.26 LOG with a single layer of Test Mesh and a net LOG reduction of 0.49 with a double layer of Test Mesh.

The foregoing descriptions of specific embodiments of the present invention have been presented for purposes of illustration and description. They are not intended to be exhaustive or to limit the invention to the precise forms disclosed, and obviously many modifications and variations are possible in light of the above teaching. The embodiments were chosen and described in order to best explain the principles of the invention and its practical application, to thereby enable others skilled in the art to best utilize the invention and various embodiments with various modifications as are suited to the particular use contemplated. It is intended that the scope of the invention be defined by the Claims appended hereto and their equivalents.

What is claimed:

1. A system including:
an anti-microbial mesh having an at least partially corrugated structure; and
a frame configured to at least partially receive a filter such that at least a portion of fluid flow through the filter interacts with the anti-microbial mesh.

2. The system of claim 1, wherein the anti-microbial mesh includes:
an anti-microbial mesh constructed at least partially from an anti-microbial material.

3. The system of claim 2, wherein the anti-microbial mesh constructed at least partially of an anti-microbial material includes:
an anti-microbial mesh constructed at least partially from an anti-microbial metal.

4. The system of claim 3, wherein the anti-microbial mesh constructed at least partially of an anti-microbial metal includes:
an anti-microbial mesh constructed at least partially from a copper-containing material.

5. The system of claim 4, wherein the anti-microbial mesh constructed at least partially of a copper-containing material includes:
an anti-microbial mesh consisting essentially of copper metal.

6. The system of claim 4, wherein the anti-microbial mesh constructed at least partially of a copper-containing material includes:
an anti-microbial mesh constructed at least partially from a copper alloy.

7. The system of claim 1, wherein the anti-microbial mesh includes:
an anti-microbial mesh including a substrate at least partially coated with an anti-microbial material.

8. The system of claim 1, wherein the frame configured to at least partially receive a filter includes:
a frame portion defining a recess configured to at partially receive the filter.

9. The system of claim 1, wherein the frame configured to at least partially receive a filter includes:
a first frame portion; and
a second frame portion.

10. The system of claim 8, wherein the first frame portion and the second frame portion include:
a first frame portion defining a recess configured to at least partially receive both the filter and the second frame portion.

11. The system of claim 1, wherein the frame configured to at least partially receive a filter includes:
a rectilinear frame supporting a substantially planar anti-microbial mesh.

12. The system of claim 1, wherein the frame configured to at least partially receive a filter includes:
a frame supporting an at least partially cylindrical anti-microbial mesh.

13. The system of claim 1, further comprising:
a filter configured to be received at least partially within the frame.

14. A system including:
an anti-microbial mesh having an open-area percentage of from 10%-50%; and
a frame configured to at least partially receive a filter such that at least a portion of fluid flow through the filter interacts with the anti-microbial mesh.

15. The system of claim 14, wherein the anti-microbial mesh having an open-area percentage of from 10%-50% includes:
anti-microbial mesh having an open-area percentage of from 20%-40%.

16. The system of claim 14, wherein the anti-microbial mesh having an open-area percentage of from 10%-50% includes:
anti-microbial mesh having an open-area percentage of approximately 30%.
